(19) United States
(12) Patent Application Publication
(10) Pub. No.: US 2014/0323897 A1
(43) Pub. Date: Oct. 30, 2014

(54) SYSTEM AND METHOD FOR ESTIMATING HIGH TIME-FREQUENCY RESOLUTION EEG SPECTROGRAMS TO MONITOR PATIENT STATE

(71) Applicants: Emery N. Brown, Brookline, MA (US); Patrick L. Purdon, Somerville, MA (US); Demba Ba, Cambridge, MA (US); Behtash Babadi, Allston, MA (US)

(72) Inventors: Emery N. Brown, Brookline, MA (US); Patrick L. Purdon, Somerville, MA (US); Demba Ba, Cambridge, MA (US); Behtash Babadi, Allston, MA (US)

(21) Appl. No.: 14/261,166

(22) Filed: Apr. 24, 2014

Related U.S. Application Data
(60) Provisional application No. 61/815,606, filed on Apr. 24, 2013.

(51) Int. Cl. A61B 5/00

(52) U.S. Cl. CPC A61B 5/4821
USPC 600/544

(57) ABSTRACT

A system and method for monitoring a patient includes a sensor configured to acquire physiological data from a patient and a processor configured to receive the physiological data from the at least one sensor. The processor is also configured to apply a spectral estimation framework that utilizes structured time-frequency representations defined by imposing, to the physiological data, a prior distributions on a time-frequency plane that enforces spectral estimates that are smooth in time and sparse in a frequency domain. The processor is further configured to perform an iteratively re-weighted least squares algorithm to perform yield a denoised time-varying spectral decomposition of the physiological data and generate a report indicating a physiological state of the patient.

### CROSS-REFERENCE TO RELATED APPLICATIONS

The present application is based on, claims priority to, and incorporates herein by reference U.S. Provisional Application Ser. No. 61/815,606, filed Apr. 24, 2013, and entitled “A METHOD FOR ESTIMATING HIGH TIME-FREQUENCY RESOLUTION EEG SPECTROGRAMS TO MONITOR GENERAL ANESTHESIA AND SEDATION.”

### STATEMENT REGARDING FEDERALLY SPONSORED RESEARCH

This invention was made with government support under 1 DPI OD003646, R01 GM104948 and DP2OD006454 awarded by the National Institutes of Health. The government has certain rights in the invention.

### BACKGROUND OF THE INVENTION

The present disclosure generally relates to systems and method for monitoring and controlling a state of a patient and, more particularly, to systems and methods for monitoring and controlling a state of a patient receiving a dose of a sedative or anesthetic compound(s) or, more colloquially, being sedated or receiving a dose of “anesthesia.”

The practice of anesthesiology involves the direct pharmacological manipulation of the central nervous system to achieve the required combination of unconsciousness, amnesia, analgesia, and immobility with maintenance of physiological stability that define general anesthesia. More that 75 years ago it was demonstrated that central nervous system changes occurring as patients received increasing doses of either ether or pentobarbital are observable via electroencephalogram (“EEG”) recordings, which measure electrical impulses in the brain through electrodes placed on the scalp. As a consequence, it was postulated that the electroencephalogram could be used as a tool to track in real time the brain states of patients under sedation and general anesthesia, the same way that an electrocardiogram (“ECG”) could be used to track the state of the heart and the cardiovascular system. Despite similar observations about systematic relationships among anesthetic doses, electroencephalogram patterns and patients' levels of arousal made by other investigators over the next several decades, use of the unprocessed electroencephalogram in real time to track the state of the brain under general anesthesia and sedation never became a standard of practice in anesthesiology.

Tools used by clinicians when monitoring patients receiving a dose of anesthesia include electroencephalogram-based (EEG) monitors, developed to help track the level of consciousness of patients receiving general anesthesia or sedation in the operating room and intensive care unit. Using proprietary algorithms that combine spectral and entropy measurements, such monitoring systems provide feedback through partial or amalgamized representations of the acquired signals, which may be used to control brain states of the patient.

Whether a patient is controlled by a system or a more traditional clinician-specific control, the results are necessarily limited by the resolution and accuracy of the underlying information that is gathered about the patient. Across nearly all fields of science and engineering, non-stationary behavior in time series data, due to evolving temporal and/or spatial dynamics, is a ubiquitous phenomenon. Common examples include speech, image and video signals, neural spike trains and electroencephalogram (EEG) measurements, seismic and oceanographic recordings, and radar emissions. Because the temporal and spatial dynamics in these time-series are often complex, non-parametric spectral techniques, rather than parametric, model-based approaches, are the methods most widely applied in the analysis of these data. Non-parametric spectral techniques based on Fourier methods, wavelets, and data-dependent approaches, such as the empirical mode decomposition (EMD), use sliding windows to take account of the non-stationarity. Although analysis with sliding windows is universally accepted, this approach has several drawbacks.

First, the spectral estimates computed in a given window do not use the estimates computed in adjacent windows, hence the resulting spectral representations do not fully capture the degree of smoothness inherent in the underlying signal. Second, the uncertainty principle imposes stringent limits on the spectral resolution achievable by Fourier-based methods within a window. Because the spectral resolution is inversely proportional to the window length, sliding window-based spectral analyses are problematic when the signal dynamics occur at a shorter time-scale than the window length. Third, in many analyses, such as EEG studies, speech processing and applications of EMD, a common objective is to compute time-frequency representations that are smooth (continuous) in time and sparse in frequency. Current spectral estimation procedures are not specifically tailored to achieve smoothness in time and sparsity in frequency. Finally, batch time series analyses are also common in many applications. Although the batch analyses can use all of the data in the recorded time-series to estimate the time-frequency representation at each time point, spectral estimation limited to local windows remains the solution of choice. Using all of the data in batch spectral analyses would enhance both time and frequency resolution.

Considering the above, there continues to be a clear need for systems and methods to accurately monitor and quantify patient states and based thereon, provide systems and methods for controlling patient states during administration of anesthetic compounds.

### SUMMARY OF THE INVENTION

The present disclosure overcomes drawbacks of previous technologies by providing systems and methods for improved spectral analysis using an iterative approach. Classical spectral estimation techniques use sliding windows to enforce temporal smoothness of the spectral estimates of non-stationary signals. This widely-adopted approach is not well suited to signals that have low-dimensional, highly-structured, time-frequency representations. Contrary to these approaches, the present disclosure provides a spectral estimation framework, termed harmonic pursuit, to compute spectral estimates that are smooth in time and sparse in frequency. A statistical interpretation of sparse recovery can be used to derive efficient algorithms for computing the harmonic pursuit spectral estimate and achieve a more precise delineation of the oscillatory structure of EEGs and neural spiking data under general anesthesia or sedation. Harmonic pursuit offers a principled alternative to existing methods for decomposing a signal into a small number of harmonic components.

In accordance with one aspect of the present disclosure, a system for monitoring a patient experiencing an administration of at least one drug having anesthetic properties is provided. The system includes at least one sensor configured to acquire physiological data from a patient, and a processor configured to receive the physiological data from the at least one sensor and assemble a time-frequency representation of signals from the physiological data. The processor is also configured to apply a state-space model for the time-frequency representation of the signals to enforce spectral estimates that are smooth in time and sparse in a frequency domain, and iteratively adjust weightings associated with the spectral estimates to converge the data toward a desired outcome. The processor is further configured to generate a report indicating a physiological state of the patient.

In accordance with another aspect of the present disclosure, a system for monitoring a patient is provided. The system includes at least one sensor configured to acquire physiological data from a patient and a processor configured to receive the physiological data from the at least one sensor, and apply a spectral estimation framework that utilizes structured time-frequency representations defined by imposing, to the physiological data, a prior distributions on a time-frequency plane that enforces spectral estimates that are smooth in time and sparse in a frequency domain. The processor is also configured to perform an iteratively re-weighted least squares algorithm to perform yield a denoised time-varying spectral decomposition of the physiological data, and generate a report indicating a physiological state of the patient.

In accordance with yet another aspect of the present disclosure, a method of processing a time-series of data is provided. The method includes applying a spectral estimation framework that utilizes structured time-frequency representations (x) of the time-series of data (y) defined by imposing, on the time-series of data, a prior distribution in a time-frequency plane that enforces spectral estimates that are smooth in time and sparse in the frequency domain and determining, using an iteratively re-weighted least squares (IRLS) algorithm, spectral estimates that are maximum a posteriori (MAP) spectral estimates. The method also includes generating a report indicating using the spectral estimates that are maximum a posteriori (MAP) spectral estimates.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will hereafter be described with reference to the accompanying drawings, wherein like reference numerals denote like elements.

FIG. 1A-B are schematic block diagrams of a physiological monitoring system.

FIG. 2 is a schematic block diagram of an example system for improved spectral analysis, in accordance with the present disclosure.

FIGS. 3A-3D are a series of estimates of spectrogram during propofol-induced general anesthesia. In particular, FIG. 3A is a spectrogram created using a single-taper estimate (12 s windows, 11 s overlap), FIG. 3B is a spectrogram created using a multitaper estimate (5 tapers), FIG. 3C is a spectrogram created using an estimate from dynamic Gaussian state-space model, and FIG. 3D is a spectrogram created using an estimate from robust state-space model in accordance with the present disclosure.

FIG. 4 is an illustration of an example monitoring and control system in accordance with the present disclosure.

FIG. 5A is a flow chart setting forth the steps of a process in accordance with the present disclosure.

FIG. 5B is another flow chart setting forth the steps of a process in accordance with the present disclosure.

FIG. 6A-6D are a series of graphs showing equivalent filters corresponding to a robust spectral estimate of example data.

FIGS. 7A-7C are a series of spectral estimates of EEG data from a subject undergoing propofol-induced general anesthesia. Specifically, FIG. 7A shows spectrograms created using a multitaper method with 2 s temporal resolution, FIG. 7B shows a spectrogram created using a multitaper method with 0.5 Hz frequency resolution, and FIG. 7C is a spectrogram created using a robust spectral estimate in accordance with the present disclosure.

FIGS. 8A-8E are a series spectral graphs showing decomposition of the neural spiking activity from a subject undergoing propofol-induced general anesthesia, where FIG. 8A shows LFP activity, FIG. 8B shows raster plot of the spike trains from the 41 units which were simultaneously acquired alongside the LFP activity, FIG. 8C shows PSTH and robust estimate of the firing rate obtained using harmonic pursuit, FIG. 8D shows spectral decomposition of the firing rate in the [0.05, 1] Hz band, and FIG. 8E shows 0.45 Hz component of log firing rate.

### DETAILED DESCRIPTION

Spectral analysis is an important tool for analyzing electroencephalogram (EEG) data. The traditional approach to clinical interpretation of the EEG is to examine time domain EEG waveforms, associating different waveform morphologies with physiology, pathophysiology, or clinical outcomes. General anesthetic and sedative drugs induce stereotyped oscillations in the EEG that are much easier to interpret when analyzed in the frequency domain using spectral analysis. Time-varying spectra are needed to track changes in drug dosage or administration, and changes in patients' level of arousal due to external stimuli. Traditional methods for nonparametric spectral estimation impose a tradeoff between time and frequency resolution. The method described in the present disclosure details an approach based on adaptive filtering and compressed sensing that can provide higher time-frequency resolution than traditional nonparametric spectral estimation methods. This method is particularly useful in estimating EEG spectrograms for monitoring general anesthesia and sedation.

As will be described, the present disclosure provides an algorithm to compute a denoised time-varying spectral decomposition of a signal. Conventional spectral estimation techniques use a combination of tapering and sliding windows to enforce smoothness and continuity of the estimated spectra. The spectral estimates using these techniques exhibit a significant amount of noise. Furthermore, although heuristics exist, selecting the size of the windows, the amount of sliding, and the number of tapers are not trivial tasks.

In one configuration, a state-space model of dynamic spectra is used that naturally promotes smoothness of the estimated spectra and performs denoising. Quantitatively, the state-space formulation leads to a principled statistical framework that uses the data to determine the optimal amount of smoothing. Qualitatively, the algorithm results in spectral estimates that are significantly sharper and less noisy than those obtained using classical spectral estimation techniques.

More formally, challenges with dynamic spectral estimation and denoising can be discussed as one of maximum a-posterior (MAP) estimation of the state sequence in a non-Gaussian state-space model. This results in a high-dimensional estimation problem for which various solutions exist. However, not all solutions can be implemented efficiently in practice. As will be described, one configuration provides a combination of Kalman smoothing techniques with an iterative re-weighting scheme to estimate denoised dynamic spectra. The algorithm is readily applicable to electroencephalogram (EEG) data collected in an operating-room (OR) setting from patients under general anaesthesia. The algorithm provides a better characterization of the neural correlates underlying various stages of general anesthesia, sedation, and related states.

### Spectral State-Space Model

Consider yₜ, t=1, . . . , T a real-valued signal; for instance the time-series recorded from a single lead of EEG. Let fₛ be the sampling frequency at which yₜ is acquired. For example, a sampling frequency common for EEG measurements is about fₛ=250 Hz, although other values are possible. A meshing/binning process can be begun at the interval [0, fₛ) to mesh into K discrete frequency bins. For example,

$${ω}_{k} = \frac{2 π \cdot k}{{f}_{s}} , \\ k = 0 , … , K - 1.$$

Note that the meshing/binning need not be uniform. Let xₜ=(x_{0,t}, x_{1,t}, . . . , x_{K-1,t})′ denote the spectrum of the signal yₜ at time t,

$${c}_{t} = {( 1 , {e}^{{jω}_{1} t} , {e}^{{jω}_{2} t} , … , {e}^{{jω}_{K - 1} t} )}^{′} . \\ {y}_{t} = {x}_{0 , t} + {\sum }_{k - 1}^{K - 1} {x}_{k , t} {e}^{\frac{j2π kt}{K}} + {v}_{t} {y}_{t} ∈ ℝ \\ {x}_{t} = {x}_{t - 1} + {w}_{t} {x}_{t} and {w}_{t} ∈ {ℝ}^{k} . \qquad ( 1 )$$

The state equation xₜ=xₜ₋₁+wₜ allows the spectrum to evolve in time in a constrained fashion. Different choices of priors on wₜ allows the capture of different characteristics of the desired spectrum, for example, abrupt changes in time, sparsity in frequency, and the like. Assume that vₜ˜N(0,1) and wₜ˜N(0,Q), where Q is a positive-definite diagonal matrix.

### Estimation of the Spectral State-Space Model

The state-space model can be rewritten compactly as:

yₜ=c′xₜ+vₜyₜε

[symbol]

xₜ=xₜ₋₁+wₜyₜ and wₜε [symbol] (2).

The denoised dynamic spectrum can be obtained by solving the following optimization problem with Q=σ² I_{K}, and I_{K} is the K×K identity matrix.

$${max}_{{x}_{1} , … , {x}_{T}} - \frac{1}{2} {( {y}_{t} - {x}_{t} )}^{2} - \frac{1}{2 {σ}^{2}} {\sum }_{t = 1}^{T} {\sum }_{k = 0}^{K - 1} {( {x}_{t , k} - {x}_{- 1 , k} )}^{2} . \qquad ( 3 )$$

The following Kalman smoothing algorithm can be used to solve for the denoised dynamic spectrum \hat{x}ₜ, t=1, . . . , T:

INPUT: Observation (yₜ)ₜ₌₁^{T}, state-noise covariance Q,
initial condition X_{0|0} and Σ_{0|0}
OUTPUT: Denoised estimate \hat{x} of time-varying spectrum
Filter:
for t ← 1 to T do
|x_{t|t - 1} = x_{t - 1|t - 1}
|Σ_{t|t - 1} = Σ_{t - 1|t - 1} +Q
|Kₜ = Σ_{t|t - 1}cₜ(cₜ^{H}Σ_{t|t - 1}cₜ + 1)⁻¹
|x_{t|t} = x_{t|t - 1} + Kₜ(yₜ − cₜ′x_{t|t - 1})
|Σ_{t|t} = Σ_{t|t - 1} − Kₜcₜ′V_{t|t - 1}
end
Smoother:
for t ← T−1 to 1 do
|Bₜ = Σ_{t|t}Σ_{t + 1|t}⁻¹
|x_{t|T} = x_{t|t} + Bₜ(x_{t + 1|T} − x_{t + 1|t})
|Σ_{t|T} = Σ_{t|t} + Bₜ(Σ_{t + 1|T} − Σ_{t + 1|t} )Bₜ^{H}
end
return \hat{x} = (x_{1|T},...,x_{T|T})′

### Robust Estimation of Dynamic Spectrum

The Kalman smoothing algorithm provided above is a significant improvement over conventional spectral estimation techniques. However, further denoising of the spectrum can be performed by solving the following optimization problem:

$${max}_{{x}_{1} , … {x}_{T}} {\sum }_{T = 1}^{T} \frac{1}{2} {( {y}_{t} - {x}_{t} )}^{2} - \frac{1}{α} {\sum }_{k = 0}^{k - 1} \sqrt{{\sum }_{t = 1}^{T} {( {x}_{t , k} - {x}_{- 1 , k} )}^{2}} . \qquad ( 4 )$$

The following novel iteratively-reweighted algorithm can be used to solve this optimization problem. Start with \hat{x}⁽⁰⁾ obtained using the above Kalman smoothing algorithm, then for l=1, . . . , 5 find:

$${\hat{x}}^{( l )} = {arg max}_{{x}_{1} , … , {x}_{T}} - \frac{1}{2} {\sum }_{t = 1}^{T} {( {y}_{t} - {x}_{t} )}^{2} - {\sum }_{t = 1}^{T} \frac{1}{2 α \sqrt{{\sum }_{t = 1}^{T} {( {\hat{x}}_{t , k}^{( l - 1 )} - {\hat{x}}_{t - 1 , k}^{( l - 1 )} )}^{2}}} {\sum }_{k = 0}^{K - 1} {( {x}_{t , k} - {x}_{t - 1 , k} )}^{2} . \qquad ( 5 )$$

If we define the diagonal matrix with entries Q_{k,k}={square root over (Σₜ₌₁^{T}(\hat{x}_{t,k}^{(l-1)}−\hat{x}_{t-1,k}^{(l-1)})²)}, k=1, . . . , K, we can express the algorithm as:

$${\hat{x}}^{( l )} = {arg max}_{{x}_{1} , … , {x}_{T}} - \frac{1}{2} {\sum }_{t = 1}^{T} {( {y}_{t} - {x}_{t} )}^{2} - \frac{1}{2 α} {\sum }_{t = 1}^{T} \frac{1}{{Q}_{k , k}} {\sum }_{k = 0}^{K - 1} {( {x}_{t , k} - {x}_{t - 1 , k} )}^{2} . \qquad ( 6 )$$

Therefore, each step of the iterative procedure can be implemented using the above-described Kalman smoothing algorithm with Q defined as above. The above-described process can be implemented using a variety of systems and for the purpose of providing useful information for a variety of clinical applications. In one configuration of the present invention, systems and methods for monitoring a state of a patient during and after administration of an anesthetic compound or compounds are provided.

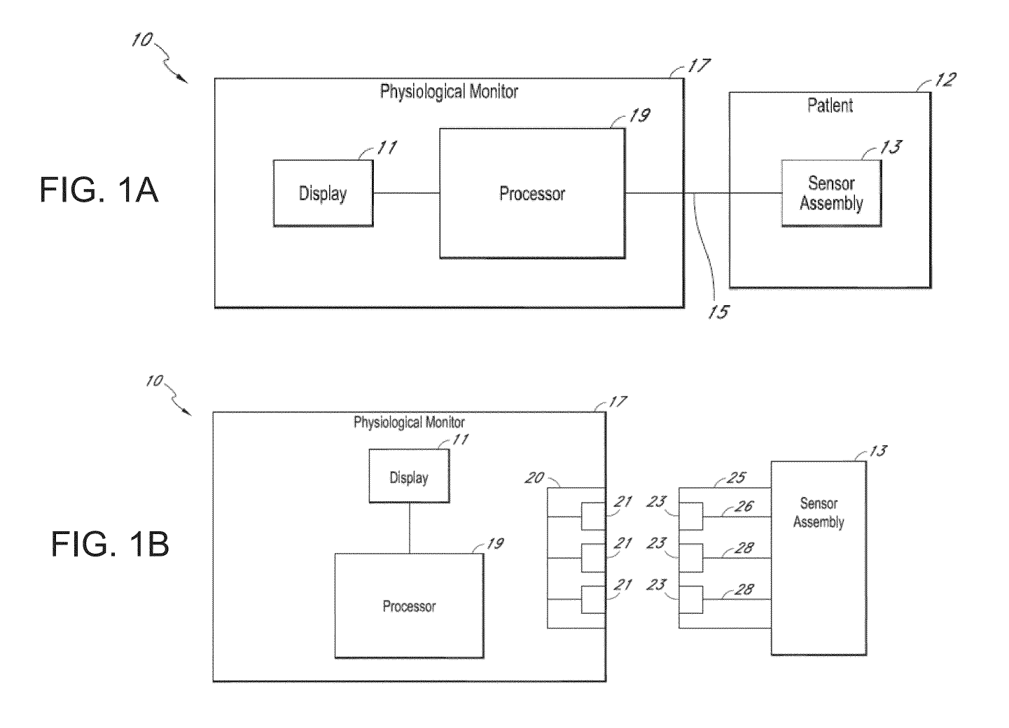

Referring specifically to the drawings, FIGS. 1A and 1B illustrate example patient monitoring systems and sensors that can be used to provide physiological monitoring of a patient, such as consciousness state monitoring, with loss of consciousness or emergence detection.

For example, FIG. 1A shows an embodiment of a physiological monitoring system 10. In the physiological monitoring system 10, a medical patient 12 is monitored using one or more sensors 13, each of which transmits a signal over a cable 15 or other communication link or medium to a physiological monitor 17. The physiological monitor 17 includes a processor 19 and, optionally, a display 11. The one or more sensors 13 include sensing elements such as, for example, electrical EEG sensors, or the like. The sensors 13 can generate respective signals by measuring a physiological parameter of the patient 12. The signals are then processed by one or more processors 19. The one or more processors 19 then communicate the processed signal to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

For clarity, a single block is used to illustrate the one or more sensors 13 shown in FIG. 1A. It should be understood that the sensor 13 shown is intended to represent one or more sensors. In an embodiment, the one or more sensors 13 include a single sensor of one of the types described below. In another embodiment, the one or more sensors 13 include at least two EEG sensors. In still another embodiment, the one or more sensors 13 include at least two EEG sensors and one or more brain oxygenation sensors, and the like. In each of the foregoing embodiments, additional sensors of different types are also optionally included. Other combinations of numbers and types of sensors are also suitable for use with the physiological monitoring system 10.

In some embodiments of the system shown in FIG. 1A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors 13.

As shown in FIG. 1B, the EEG sensor 13 can include a cable 25. The cable 25 can include three conductors within an electrical shielding. One conductor 26 can provide power to a physiological monitor 17, one conductor 28 can provide a ground signal to the physiological monitor 17, and one conductor 28 can transmit signals from the sensor 13 to the physiological monitor 17. For multiple sensors, one or more additional cables 15 can be provided.

In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor 13 and the physiological monitor 17 communicate wirelessly.

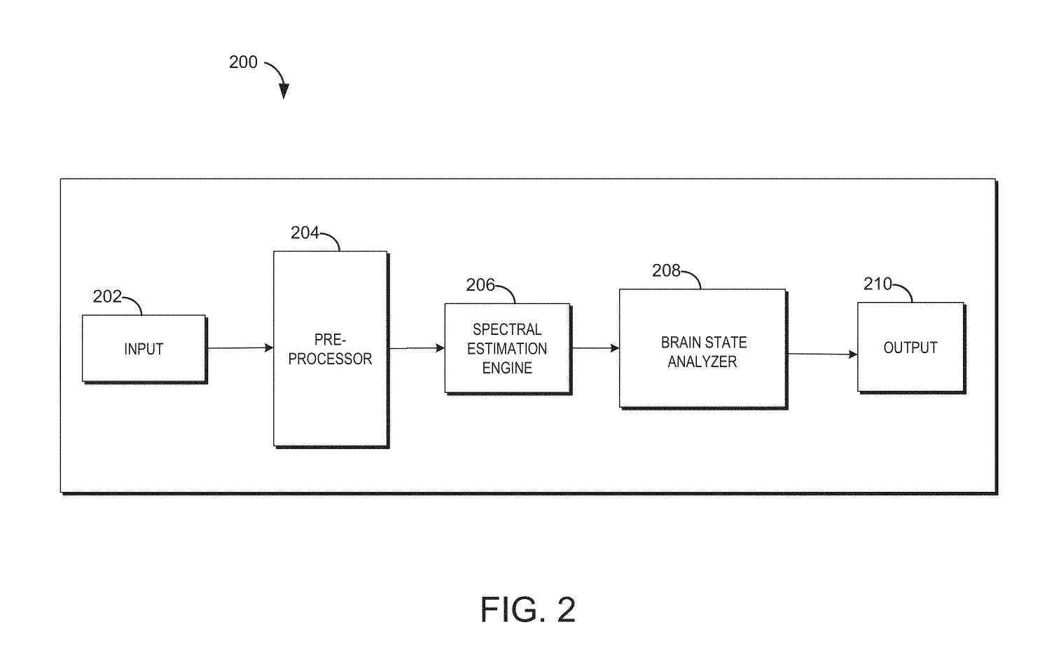

Referring to FIG. 2, an example system 200 for use in carrying out steps associated with determining a state of a brain patient using an improved spectral estimation approach, as described above, is illustrated. The system 200 includes an input 202, a pre-processor 204, a spectral estimation engine 206, a brain state analyzer 208, and an output 210. Some or all of the modules of the system 200 can be implemented by a physiological patient monitor as described above with respect to FIG. 1.

The pre-processor 204 may be designed to carry out any number of processing steps for operation of the system 200. In addition, the pre-processor 204 may be configured to receive and pre-process data received via the input 202. For example, the pre-processor 204 may be configured to assemble a time-frequency representation of signals from the physiological data, such as EEG data, acquired from a subject, and perform any desirable noise rejection to filter any interfering signals associated with the data. The pre-processor 204 may also be configured to receive an indication via the input 202, such as information related to administration of an anesthesia compound or compounds, and/or an indication related to a particular patient profile, such as a patient's age, height, weight, gender, or the like, as well as drug administration information, such as timing, dose, rate, and the like.

In addition to the pre-processor 204, the system 200 may further include a spectral estimation engine 206, in communication with the pre-processor 202, designed to receive pre-processed data from the pre-processor 202 and carry out steps necessary for a spectral analysis that applies a state-space model for a time-frequency representation of signals to enforce spectral estimates that are smooth in time and sparse in a frequency domain. As a result, the spectral estimation engine 206 may provide denoised time-varying spectral decompositions of acquired physiological signals, which may then be used by the brain state analyzer 208 to determine brain state(s), such as a state of consciousness or sedation, of a patient under administration of a drug with anesthetic properties, as well as confidence indications with respect to the determined state(s). Information related to the determined state(s) may then be relayed to the output 210, along with any other desired information, in any shape or form. In some aspects, the output 210 may include a display configured to provide information or indicators with respect to denoised spectral decompositions, that may be formulated using spectrogram representations, either intermittently or in real time.

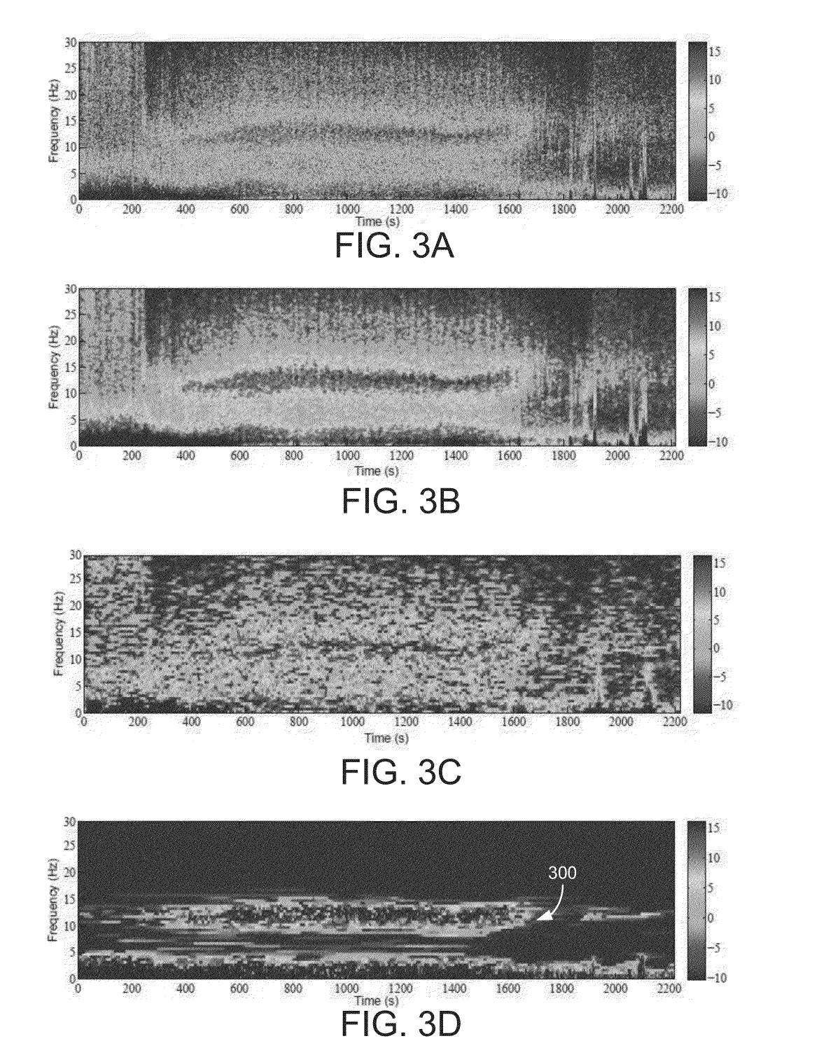

For example, FIGS. 3A through 3D provide a series of spectrograms created using different methods and illustrating the substantially-improved results of using the above-described systems and methods. In particular, FIG. 3A shows a spectrogram created using a single-taper estimate (12 s windows, 11 s overlap). On the other hand, FIG. 3B shows a spectrogram created using multitaper estimate (5 tapers). Additionally, FIG. 3C shows a spectrogram created using a dynamic Gaussian state-space model. Finally, FIG. 3D shows a spectrogram created using the above-described, robust state-space approach. As can be readily seen, noise is greatly reduced in FIG. 3D compared to the other spectrograms and the salient information 300, such as power evolution in a frequency range say, between 10 Hz and 15 Hz, is highly-discernible.

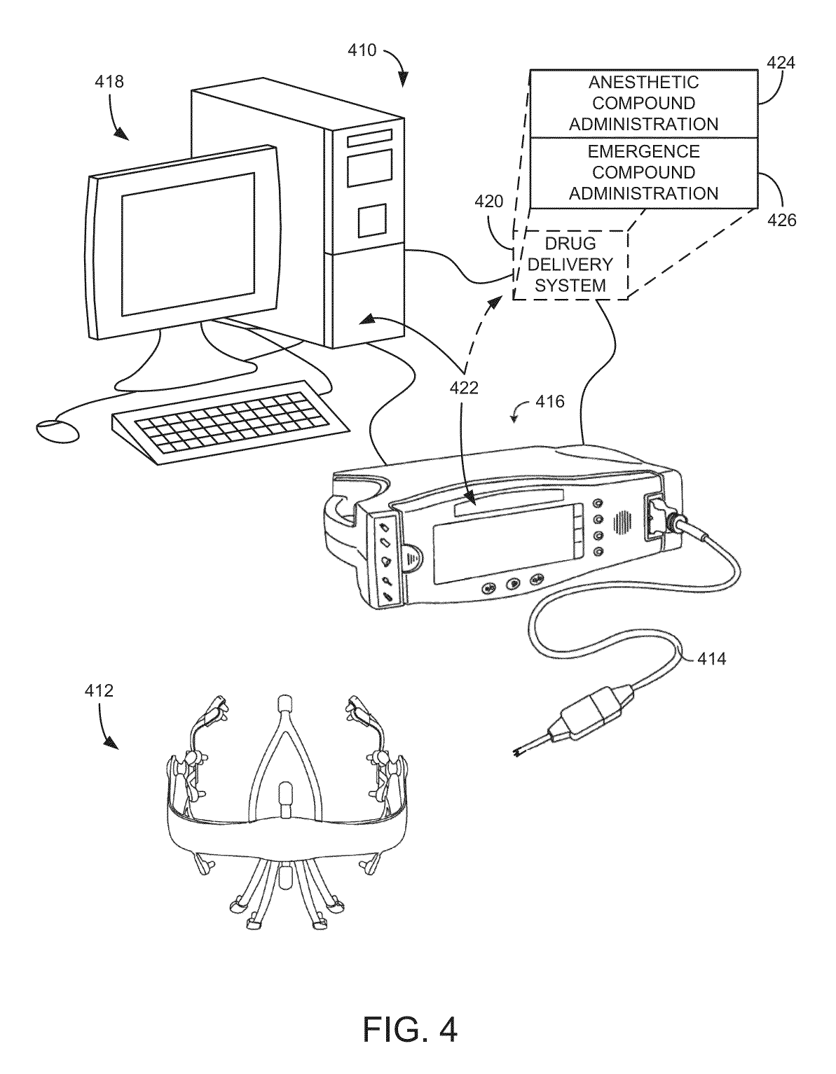

Referring to FIG. 4, an system 410 in accordance with one aspect the present invention is illustrated. The system 410 includes a patient monitoring device 412, such as a physiological monitoring device, illustrated in FIG. 4 as an electroencephalography (EEG) electrode array. However, it is contemplated that the patient monitoring device 412 may also include mechanisms for monitoring galvanic skin response (GSR), for example, to measure arousal to external stimuli or other monitoring system such as cardiovascular monitors, including electrocardiographic and blood pressure monitors, and also ocular Microtremor monitors. One specific configuration of this design utilizes a frontal Laplacian EEG electrode layout with additional electrodes to measure GSR and/or ocular microtremor. Another configuration of this design incorporates a frontal array of electrodes that could be combined in post-processing to obtain any combination of electrodes found to optimally detect the EEG signatures described earlier, also with separate GSR electrodes. Another configuration of this design utilizes a high-density layout sampling the entire scalp surface using between 64 to 256 sensors for the purpose of source localization, also with separate GSR electrodes.

The patient monitoring device 412 is connected via a cable 414 to communicate with a monitoring system 416. Also, the cable 414 and similar connections can be replaced by wireless connections between components. As illustrated, the monitoring system 416 may be further connected to a dedicated analysis system 418. Also, the monitoring system 416 and analysis system 418 may be integrated.

The monitoring system 416 may be configured to receive raw signals acquired by the EEG electrode array and assemble, and even display, the raw signals as EEG waveforms. Accordingly, the analysis system 418 may receive the EEG waveforms from the monitoring system 416 and, as will be described, process the EEG waveforms and generate a report, for example, as a printed report or, preferably, a real-time display of information. However, it is also contemplated that the functions of monitoring system 416 and analysis system 418 may be combined into a common system. In one aspect, the monitoring system 416 and analysis system 418 may be configured to determine a current and future brain state under administration of anesthetic compounds, such as during general anesthesia or sedation.

The system 410 may also include a drug delivery system 420. The drug delivery system 420 may be coupled to the analysis system 418 and monitoring system 416, such that the system 410 forms a closed-loop monitoring and control system. Such a closed-loop monitoring and control system in accordance with the present invention is capable of a wide range of operation, but includes user interfaces 422 to allow a user to configure the closed-loop monitoring and control system, receive feedback from the closed-loop monitoring and control system, and, if needed, reconfigure and/or override the closed-loop monitoring and control system.

In some configurations, the drug delivery system 420 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness.

For example, in accordance with one aspect of the present invention, methylphenidate (MPH) can be used as an inhibitor of dopamine and norepinephrine reuptake transporters and actively induces emergence from isoflurane general anesthesia. MPH can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. Also, ethylphenidate or other agents can be used to actively induce emergence from isoflurane, propofol, or other general anesthesia by increasing arousal using a control system, such as described above. For example, the following drugs are non-limiting examples of drugs or anesthetic compounds that may be used with the present invention: Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and the like.

Therefore, a system, such as described above with respect to FIG. 4, can be provided to carry out active emergence from anesthesia by including a drug delivery system 420 with two specific sub-systems. As such, the drug delivery system 420 may include an anesthetic compound administration system 424 that is designed to deliver doses of one or more anesthetic compounds to a subject and may also include a emergence compound administration system 426 that is designed to deliver doses of one or more compounds that will reverse general anesthesia or the enhance the natural emergence of a subject from anesthesia.

Using the concepts and models provided above, as applied, for example, to EEG data acquired with a system such as described above, a process for non-parametric spectral analysis for batch time series as a Bayesian estimation problem is provided by introducing prior distributions on the time-frequency plane. As will be described, the process can be used to yield maximum a posteriori (MAP) spectral estimates that are continuous in time yet sparse in frequency. This spectral estimation procedure, termed harmonic pursuit, can be efficiently computed using an iteratively re-weighted least squares (IRLS) algorithm and scales well with typical data lengths. Harmonic pursuit works by applying to the time series a set of data-derived filters. Using a link between Gaussian mixture models, l₁ minimization and an Expectation-Maximization algorithm, it will be shown that the harmonic pursuit algorithm converges to the global MAP estimate.

Begin with a toy example to highlight the deficiencies of classical techniques in analyzing non-stationary time-series and the limits they impose on spectral resolution. In this case, noisy observations can be simulated from the linear combination of two amplitude-modulated signals as:

$${y}_{t} = 10 {cos}^{8} ( 2 π {f}_{0} t ) sin ( 2 π {f}_{1} t ) + 10 exp ( 4 \frac{t - T}{T} ) cos ( 2 π {f}_{S} t ) + {v}_{t} , \\ for \\ 0 \leq t \leq T , ; \qquad ( 7 )$$

where ƒ₀=0.04 Hz, ƒ₁=10 Hz, ƒ₂=11 Hz, T=600 s, and (vₜ)ₜ₌₁^{T} is independent, identically-distributed, zero-mean Gaussian noise with variance set to achieve a signal-to-noise ratio (SNR) of 5 dB. The simulated data can consist of a 10 Hz oscillation whose amplitude is modulated by a slow 0.04 Hz oscillation, and an exponentially growing 11 Hz oscillation. The former is motivated by the fact that low-frequency (<1 Hz) phase modulates alpha (8-12 Hz) amplitude during profound unconsciousness, and during the transition into and out of unconsciousness, under propofol-induced general anesthesia. The latter can be incorporated to demonstrate the desire, in certain applications, to resolve closely-spaced amplitude-modulated signals.

This toy example poses serious challenges for classical spectral estimation algorithms due to the strong amplitude modulation and non-stationarities, observation noise, and identifiability issues (the decomposition is not unique). However, as will be described, the above-described process can be extended to provides an analysis paradigm to separate signals such as the 10 and 11 Hz oscillations and recover the modulating processes. This spectral estimation framework utilizes the concept of structured time-frequency representations defined by imposing a prior distribution on the time-frequency plane, which enforces spectral estimates that are smooth in time, yet sparse in the frequency domain. The high-dimensional estimation problem can be solved by using an efficient iteratively re-weighted least squares algorithm.

As described above, consider again yₜ, a real-valued signal, for instance the time-series recorded from a single lead of EEG, where t=1, 2, . . . , T. The signal may be obtained by sampling of the underlying, noise-corrupted, continuous-time signal at a rate fₛ (above the Nyquist rate). Given an arbitrary interval of length W, let yₙ:=(y_{(n-1)W+1}, y_{(n-1)W+2}, . . . , y_{nW})′ for n=1, 2, . . . N with

$$N \text{:} = \frac{T}{W} .$$

Without loss of generality, assume that T is an integer multiple of W and consider the following dynamic harmonic representation of yₙ as:

yₙ=\tilde{F}ₙ\tilde{x}ₙ+vₙ (8);

where

$${( {\tilde{F}}_{n} )}_{l , k} \text{:} = exp ( j 2 π ( ( n - 1 ) W + l ) \frac{k}{K} ,$$

with l=1, 2, . . . , W, k=0, 1, . . . , K−1, and \tilde{x}ₙ:=(\tilde{x}_{n,0}, \tilde{x}_{n,1}, . . . , \tilde{x}_{n,K})′ε [symbol] ^{K} with K being a positive integer, and vₙ is independent, identically-distributed, additive zero-mean Gaussian noise. Each observation window of length W is considered to have a discrete Cramer representation \tilde{x}ₙ of dimension K. Equivalently, the Cramer representation can be defined over a real vector space as xₙε [symbol] ^{K}, with the equivalent observation model:

yₙ=Fₙxₙ+vₙ (9);

where

$${( {F}_{n} )}_{l , k} \text{:} = cos ( j 2 π ( ( n - 1 ) W + l ) \frac{k}{K} )$$

$${and \\ ( {F}_{n} )}_{l , k + K \text{/} 2} \text{:} = sin ( j 2 π ( ( n - 1 ) W + l ) \frac{k + K \text{/} 2}{K} )$$

for l=1, 2, . . . , W and

$$k = 0 , 1 , … , \frac{K}{2} - 1 ,$$

and xₙ:=(x_{n,1}, x_{n,1}, . . . , x_{n,K})′ε [symbol] ^{K}. Equation (9) can be conveniently re-written in vector form as Fx+v, where F is a T×NK block-diagonal matrix with Fₙ on the diagonal blocks:

$$F \text{:} = ( \begin{matrix}{F}_{1} & & & \\ & {F}_{2} & & \\ & & … & \\ & & & {F}_{N}\end{matrix} ) ; \qquad ( 10 )$$

where y=(y₁, y₂, . . . , y_{T})′ε [symbol] ^{T}, x=(x′₁, x′₂, . . . , x′_{N})′ε [symbol] ^{NK}, and v=(v′₁, v′₂, . . . , v′_{N})′ε [symbol] ^{T}. Here, x can be viewed as a time-frequency representation of the non-stationary signal y. This linear-Gaussian forward model can be generalized to nonlinear harmonic parameterizations of the joint distribution of non-Gaussian data.

The objective is to compute an estimate of x, represented as \hat{x}, given the data y. The component-wise magnitude-squared of \hat{x}ε [symbol] ^{NK} then gives the power spectral density of y. Classical spectral estimation techniques use sliding windows with overlap to implicitly enforce temporal smoothness, but they do not consider sparsity in the frequency domain. In contrast, a direct approach, as provided herein, which treats (xₙ)ₙ₌₁^{N} as a random process and explicitly imposes a stochastic continuity constraint on its elements across time, as well as a sparsity constraint across frequency. By construction, xₙ is the discrete Cramer representation of yₙ. If (yₙ)ₙ₌₁^{N} were a stationary process, (xₙ)ₙ₌₁^{N} would be an independent discrete orthogonal process. However, such a process does not account for temporal smoothness or sparsity in the frequency domain. To this end, temporal smoothness can be achieved by modeling the dependence between xₙ and xₘ for m≠n. Similarly, imposing a model on the components (xₙ)ₖ₌₁^{K} for each n=1, 2, . . . , N can enforce sparsity in the frequency domain. Starting with a known initial condition x₀, the stochastic continuity constraint takes the form:

xₙ=xₙ₋₁+wₙ, (11);

where w=(w′₁, w′₂, . . . , w′_{N})ε [symbol] ^{NK} is a generic random process. In other words, it is assumed that the discrete Cramer representation of the data follows a first-order difference equation. To enforce temporal smoothness and sparsity in frequency domain, a joint prior probability density function is enforced over w₁, w₂, . . . , w_{N}, which in turn imposes a joint probability density function on the set of discrete Cramer representations (xₙ)ₙ₌₁^{N}, or equivalently on the time-frequency plane. Motivated by the empirical mode decomposition and its variants, prior densities can be chosen that enforce sparsity in the frequency domain and smoothness in time. In logarithmic form, proposed priors include:

$$\begin{matrix}log {p}_{1} ( {w}_{1} , {w}_{2} , … , {w}_{N} ) ∝ α {\sum }_{k = 1}^{K} {( {\sum }_{n = 1}^{N} {ω}_{n , k}^{2} + {∈}^{2} )}^{\frac{1}{2}} ; \\ and & ( 12 ) \\ log {p}_{2} ( {w}_{1} , {w}_{2} , … , {w}_{N} ) ∝ α {\sum }_{k = 1}^{K} {( {\sum }_{n = 1}^{N} {( {ω}_{n , k}^{2} + {∈}^{2} )}^{\frac{1}{2}} )}^{\frac{1}{2}} ; & ( 13 )\end{matrix}$$

where α>0 and ε>0 is a small constant. These priors belong to the Gaussian scale mixture (GSM) family of densities. This family of densities has robustness properties in the statistical sense. Both p₁(•) and p₂(•) enforce inverse solutions which are spatially sparse and temporally smooth. Unlike p₁(•), p₂(•) can capture abrupt changes in the dynamics of the inverse solution. Under each of these priors, the discrete-time stochastic process (xₙ)ₙ₌₁^{N} is a non-Gaussian random process whose increments are statistically dependent. Together, equations (9) and (11) form a state-space model for the time-frequency representation of the signal.

### The Inverse Solution: Harmonic Pursuit.

The problem of robust spectral estimation can be formulated as one of Bayesian estimation in which the posterior density of x given y fully characterizes the space of inverse solutions. The forward model of equation (9) specifies the likelihood of the data, that is to say the conditional density of y given x. Assuming that the observation noise vₙ are samples from independent, identically-distributed, zero-mean Gaussian random vectors with covariance σ²I. To simplify the notation, let:

ƒᵢ(x₁,x₂, . . . x_{N}):=log pᵢ(x₁−x₀,x₂−x₁, . . . x_{N}−x_{N-1}) (14);

for i=1 and 2 in what follows. Robust spectral estimates can be computed by solving the maximum a posteriori (MAP) estimation problem:

$${max}_{{x}_{1} , … , {x}_{N}} - {\sum }_{n = 1}^{N} \frac{1}{2 {σ}^{2}} {‖ {y}_{n} - {F}_{n} {x}_{n} ‖}_{2}^{2} - f ( {x}_{1} , {x}_{2} , … , {x}_{N} ) . ; \qquad ( 15 )$$

The MAP estimation problem of equation (15) is referred to herein as the harmonic pursuit problem, and its solution the harmonic pursuit estimate. To solve this optimization problem, the constant σ in f(•) (specifically in a from equations (12) and (13). Therefore, henceforth, assume that σ=1.

Equation (15), with f(•)=f₁(•), is a strictly concave optimization problem which, in principle, can be solved using standard techniques. However, experience has shown that these techniques do not scale well with N because of the batch nature of the problem. The solution to \hat{x} to equation (15) can be obtained as the limit of a sequence (\hat{x}^{(l)})ₗ₌₀^{∞} whose l^{th} element l=1, . . . , ∞, is the solution to a Gaussian MAP estimation problem (constrained least squares program) of the form:

$${max}_{{x}_{1} , … , {x}_{N}} - {\sum }_{n = 1}^{N} \frac{1}{2 {σ}^{2}} {‖ {y}_{n} - {F}_{n} {x}_{n} ‖}_{2}^{2} - {\sum }_{k = 1}^{K} {\sum }_{n = 1}^{N} \frac{ {( {x}_{n , k} - {x}_{n - 1 , k} )}^{2}}{2 {( {Q}_{n}^{( 𝓁 )} )}_{k , k}} . \qquad ( 16 )$$

For each l,Qₙ^{(l)} is a K×K diagonal matrix which depends on α, \hat{x}ₙ₋₁^{(l-1)}, \hat{x}ₙ^{(l-1)} and f(•). For instance, for f(•)=f1(•), it follows:

$${( {Q}^{( 𝓁 )} )}_{k , k} = \frac{{( {\sum }_{n = 1}^{N} {( {\hat{x}}_{n , k}^{( 𝓁 - 1 )} - {\hat{x}}_{n - 1 , k}^{( 𝓁 - 1 )} )}^{2} + {∈}^{2} )}^{\frac{1}{2}}}{α} ; \qquad ( 17 )$$

Equation (16) is a quadratic program with strictly concave objective and block-tridiagonal Hessian. The Fixed Interval Smoother, such as described in Rauch H, Striebel C, Tung F (“Maximum likelihood estimates of linear dynamic systems”, AIAA journal 3, 2012) and incorporated herein by reference in its entirety, exploits this structure to give an efficient solution to this program via forward-backward substitution. The following example summarizes this iterative solution to the harmonic pursuit problem in accordance with the present disclosure.

### Harmonic Pursuit Algorithm

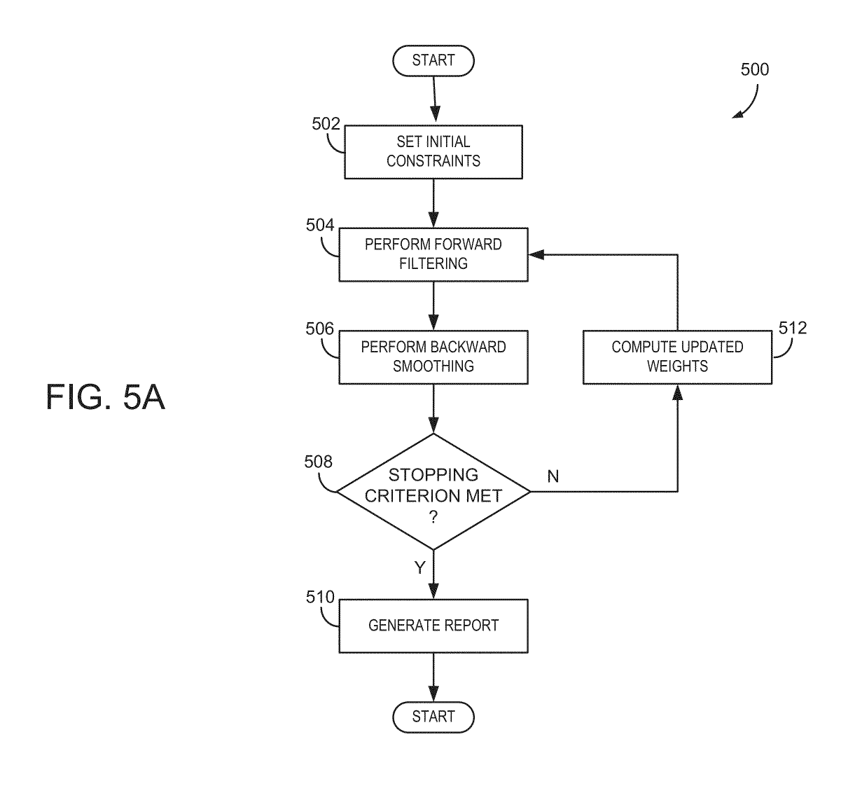

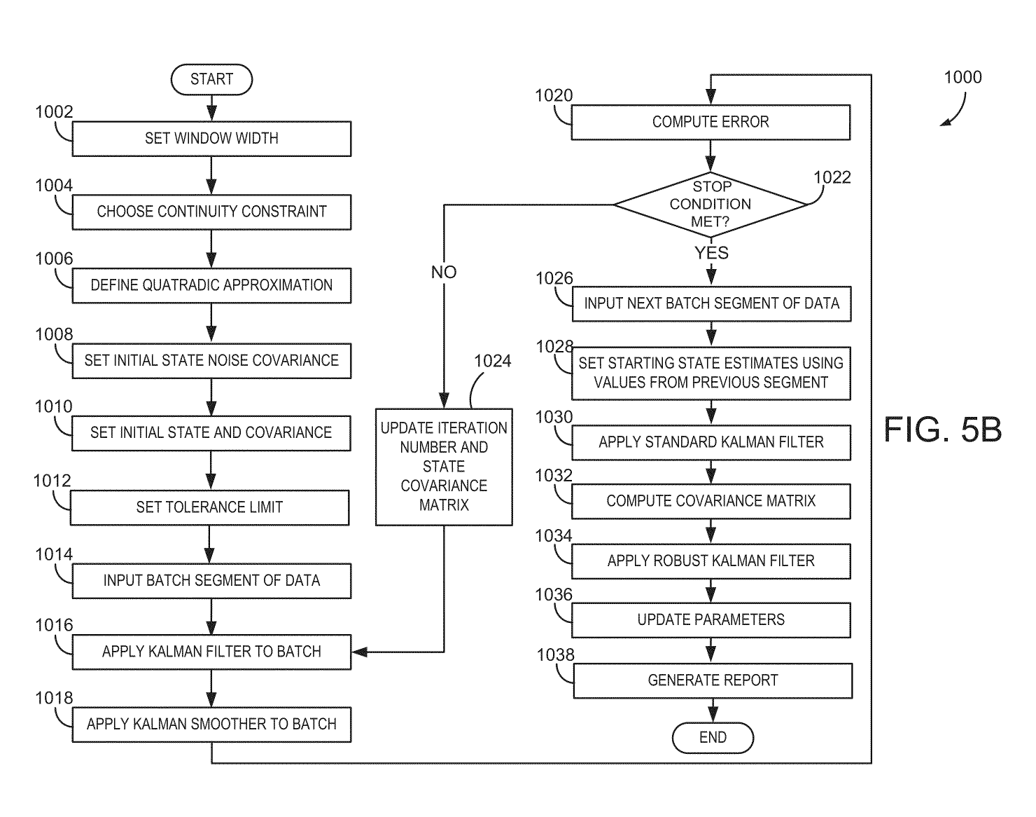

Referring to FIG. 5A, a flow chart is provided that sets forth steps of iterative a solution to the harmonic pursuit problem in accordance with the present disclosure. The process 500 starts by setting initial constraints at process block 502. Namely, the inputs are observations y, initial guess \hat{x}⁽⁰⁾ of solution, state-noise covariances Qₙ⁽⁰⁾, n=1, . . . , N, initial conditions x_{0|0}, Σ_{0|0}, tolerance tolε(0,0.01), and maximum number of iterations Lₘₐₓε [symbol] ⁺. Before moving into the iterative process that follows, the iteration number l is initialized to 1.

At process block 504, a forward filtering is performed. Specifically, at time n=1, 2, . . . , N, filter:

x_{n|n-1}=x_{n-1|n-1}

Σ_{n|n-1}=Σ_{n-1|n-1}+Qₙ^{(l-1)}

Kₙ=Σ_{n|n-1}Fₙ^{H}(FₙΣ_{n|n-1}Fₙ^{H}+σ²I)⁻¹

x_{n|n}=x_{n|n-1}+Kₙ(yₙ−Fₙx_{n|n-1})

Σ_{n|n}=Σ_{n|n-1}−KₙFₙΣ_{n|n-1}

Then, at process block 506, a backward smoothing is performed. In particular, at time n=N−1, N−2, . . . , 1, smoother:

$${B}_{n} = {\sum }_{n \vert n} {\sum }_{n + 1 \vert n}^{- 1} \\ {x}_{n \vert N} = {x}_{n \vert n} + {B}_{n} ( {x}_{n + 1 \vert N} - {x}_{n + 1 \vert n} )$$

$$Let {\hat{x}}_{n}^{( 𝓁 )} = {x}_{n \vert N} , n = 1 , … , N and {\hat{x}}^{( 𝓁 )} = {( {\hat{x}}_{1}^{{( 𝓁 )}^{′}} , … , {\hat{x}}_{N}^{{( 𝓁 )}^{′}} )}^{′} .$$

At decision block 508, the stopping criterion is checked. Namely, if

$$\frac{{‖ {\hat{x}}^{( 𝓁 )} - {\hat{x}}^{( 𝓁 - 1 )} ‖}_{2}}{{‖ {\hat{x}}^{( 𝓁 - 1 )} ‖}_{2}} < tol or 𝓁 = {L}_{max} ,$$

a report is generated at process block 510 and the process ends. Else, at process block 512, the weights are updated by letting l=l+1 and updating the state covariance Q^{(l)}:

$${( {Q}^{( 𝓁 )} )}_{k , k} = \frac{{( {\sum }_{n = 1}^{N} {( {\hat{x}}_{n , k}^{( 𝓁 - 1 )} - {\hat{x}}_{n - 1 , k}^{( 𝓁 - 1 )} )}^{2} + {∈}^{2} )}^{\frac{1}{2}}}{α}$$

The output indicates \hat{x}^{(L)}, where L≦Lₘₐₓ is the number of the last iteration of the algorithm.

The harmonic pursuit algorithm is an instance of iteratively reweighted least squares (IRLS) algorithms. For f(•)=f1(•), starting with \hat{x}⁽⁰⁾ a guess of the solution, equation (16) can be solved for l=1, 2, . . . , L with Q^{(l)} iteratively updated using equation (17). L is the smallest of Lₘₐₓ, a pre-specified maximum number of iterations, and the number of iterations when the convergence criterion of decision block 508 of the above-described harmonic pursuit algorithm is first satisfied. Consistent with previous reports, a small number of IRLS iterations (between 5 and 10) was found to be sufficient in practice. Due to the dynamic nature of our state-space model, the MAP estimation problem in equation (16) can be solved using an iterative solution given by the fixed interval smoother. For the penalization function f1(•), Q^{(l)} is independent of n.

Solving the IRLS problem in equation (16) with (Q^{(l)})_{k,k} in equation (17) is an EM algorithm for solving equation (12) with f(•)=f1(•). The EM algorithm minorizes the objective function of equation (15) by a local quadratic approximation to the log-prior, which results in the least squares problem of equation (16). The Hessian of the quadratic approximation is given by Q^{(l)} in equation (17). One way to derive equation (17) is to invoke the concavity of the function s^{1/2}, which implies that the linear approximation around a point s̅ satisfies

$${s}^{1 / 2} \leq {\overline{s}}^{1 / 2} + \frac{1}{2 {\overline{s}}^{1 / 2}} ( s - \overline{s} ) .$$

Applying this result to log-prior at \hat{x}^{(l-1)} allows ready extraction of (Q^{(l)})_{k,k} in equation (17).

In general, it can be shown that for priors from the Gaussian scale mixtures family of distributions, the EM algorithm for solving the MAP problem of equation (16) results in IRLS solutions. The connection to EM theory leads to a class of IRLS algorithms much broader than that considered in the existing literature and a much simpler convergence analysis. Convergence of the IRLS algorithm of equation (16) is established in Theorem 1, as follows.

Theorem 1. Let \hat{x}⁽⁰⁾ε [symbol] ^{K} and (\hat{x}^{(l)})ₗ₌₁^{∞}ε [symbol] ^{NK} be the sequence generated by the IRLS algorithm of equation (16) with (Q^{(l)})_{k,k} as in equation (17). Then, (i) (\hat{x}^{(l)})ₗ₌₀^{∞} is bounded, (ii)

$$∃ \overline{x} = {lim}_{l \to ∞} {\hat{x}}^{( l )} ,$$

and (iii) (\hat{x}^{(l)})ₗ₌₀^{∞} converges to the unique stationary point of the objective of equation (15) with f(•)=f₁(•).

The proof follows the same lines as that of Theorem 3 in Ba D, Babadi B, Purdon P, Brown E (2013). Convergence and stability of iteratively reweighted least squares algorithms, to appear in IEEE Transactions on Signal Processing., which is incorporated herein by reference in its entirety. Thus, only the main ideas are presented here.

Proof 1. Let f(•)=f₁(•) in equation (15). The concavity of the \sqrt{•} function implies that the objective in equation (16) with (Q^{(l)})_{k,k} as in equation (17) is a lower bound of that in equation (15). Moreover, the difference between these two objectives attains a maximum at \hat{x}^{(l)}. This can be used to show that equation (16) generates a sequence that is non-decreasing when evaluated at the objective of equation (15). Along with the fact that the objective of equation (15) is strictly concave and coercive, this implies that the sequence (\hat{x}^{(l)})ₗ₌₀^{∞} lies in a compact set, and hence is bounded. Therefore, there exists a convergent subsequence with limit point x̅. Take any convergent subsequence, each of its elements satisfies the first-order necessary and sufficient optimality conditions for the objective of equation (16) which, in the limit, are equivalent to the first-order necessary and sufficient optimality condition for the unique maximizer of equation (15).

Convergence of the IRLS algorithm of equation (16) does not follow from the analysis of Daubechies and colleagues, as described in Daubechies I, DeVore R, Fornasier M, G\ddot{}unt \ddot{}urk C (2009) Iteratively reweighted least squares minimization for sparse recovery. Communications on Pure and Applied Mathematics 63:1-38. The proof requires novel ideas that we adapt to the current setting.

The difficulty in solving the harmonic pursuit problem of equation (15) arises from the choice of the prior probability density functions over w₁, w₂, . . . , w_{N} equations (12) and Eq. (13) which, for each k=1, . . . K, groups (w_{n,k})ₙ₌₁^{N} across time. At first glance, this grouping suggests a batch solution. However, recognizing the connection with the EM theory for Gaussian scale mixtures yields an IRLS solution that can be solved efficiently using the fixed interval smoother, owing to the dynamic structure of the state-space model.

Specialize the discussion above to f(•)=f2(•), let:

$${( {Q}^{( 𝓁 )} )}_{k , k} = \frac{2}{α} {( {( {\hat{x}}_{n , k}^{( 𝓁 - 1 )} - {\hat{x}}_{n - 1 , k}^{( 𝓁 - 1 )} )}^{2} + {∈}^{2} )}^{\frac{1}{2}} \times {( {{\sum }_{{n}^{′} = 1}}^{N} {( {( {\hat{x}}_{{n}^{′} , k}^{( 𝓁 - 1 )} - {\hat{x}}_{{n}^{′} - 1 , k}^{( 𝓁 - 1 )} )}^{2} + {∈}^{2} )}^{\frac{1}{2}} )}^{\frac{1}{2}} . \qquad ( 18 )$$

Equation (16) becomes an IRLS algorithm for solving the harmonic pursuit problem with f(•)=f₂(•). In this case, the convergence of the algorithm can also be proven. However, since f₂(•) is not convex, convergence can only be guaranteed to a stationary point of the objective in equation (15). In both cases, the role of ε is to avoid division by zero in forming the quadratic approximation of equation (16).

As described, the terminology of a robust spectral estimator and estimate is used to refer to harmonic pursuit and its solution, respectively. The former terminology reflects the fact that f₁(•) and f₂(•) correspond to Gaussian Scale Mixture prior distributions on x₁, x₂−x₁, . . . , x_{N}x_{N-1}, which are robust in the statistical sense.

Referring particularly to FIG. 5B, a process 1000 in accordance with the present disclosure is provided. The process 1000 starts with process block 1002 where an observation window width is set, followed by choosing the form of the continuity constraint at process block 1004 and defining the quadratic approximation to the continuity constraint at process block 1006. Then, the initial state noise covariance is set at process block 1008, the initial state and covariance estimate are set at process block 1010, and the tolerance limit for convergence of the batch algorithm is set at process block 1012. Input of the initial batch segment of data is performed at process block 1014 followed by application of the Kalman filter and the Kalman smoother algorithms to the batch segment data at process block at process block 1016 and 1018.

Subsequently, at process block 1020 an error value is computed as the relative magnitude of the difference in the state variables between a previous and a current iteration. At decision block 1022 the algorithm if the error is less than the tolerance or the maximum iteration is reached, and if not, the iteration number and new state covariance matrix are updated, and the process 1000 repeats from process block 1016. In certain configurations, the algorithm may be terminated at condition block 1022 and the state (spectral) estimates from the batch data is provided as output. Such output may be in the form of a displayed representation, such as a spectrogram representation. Thus, if parameter estimates are performed via process block 1002-1020 carried out once, an update to a spectrogram can be achieved around every second.

At process block 1026 a subsequent segment of data is provided as input, followed by a step of setting the state estimates computed from the end of the previous segment as starting values for the state estimation at process block 1028. At process block 1030 a standard Kalman filter algorithm may be applied to the data, with computation of the covariance matrix for the robust Kalman filter from the Kalman filtered state estimates then performed at process block 1032. At process block 1034 the robust Kalman filter may be applied the data to obtain the robust spectral estimates. In some aspects, the Kalman filter and robust Kalman filter algorithms may be performed in parallel to produce the robust spectral estimates. As data accrue, at process block 1036, parameters of the robust smoother may be updated via process blocks 1002-1020. In this manner, the robust Kalman filter may be applied until all data desired processing, by computation of robust spectral estimates, is complete. Finally, at process block 1038, a report, of any shape or form, is generated. The report may be representative of (spectral) estimate information acquired by way of steps described, for example, and displayed using a spectrogram representation.

Robust spectral estimates, generated as described, may be relayed to any systems configured to receive and apply such information for a variety of applications. For example, robust spectral information, obtained in a manner provided by the present disclosure, may facilitate identification of drug-specific signatures, states of consciousness, and anesthesia. The high-precision frequency structure identified using methods described could be used to identify specific anesthetic drugs by comparing the harmonic structure to those of known anesthetic drugs or combinations of drugs. In particular, a robust spectral analysis may identify different frequency bands depending on the anesthetic being used. For example, in the case of propofol, a spectral analysis using spectral estimates provided may include slow-delta oscillations and alpha oscillations identification and characterization. Similarly, for dexmedetomidine a spectral analysis could include slow-delta oscillations and spindle oscillations, while for low ketamine and high-dose ketamine, a spectral analysis could include high beta and low gamma oscillations, and slow-delta oscillations, respectively. In addition, for sevoflurane, desflurane and isoflurane an analysis could slow-delta oscillations, theta and alpha oscillations

Similarly, a harmonic structures provided could be used to identify different drug-induced states of altered consciousness or sedation, or to identify different levels of anesthesia compatible with different levels of surgical stimuli.

In some aspects, implementing the above algorithm, as described, in systems configured to display spectral information, for example, in the form of spectrograms, such systems may be capable of providing updates of displayed spectrogram, say every five-seconds. However, if the parameter estimates in steps 1 to 14 are carried out only once the update to the spectrogram can be carried out every second.

Using methods described, a high spectral resolution is achievable because the robust spectral analysis identifies power in a sparse (small) set of frequency bands. The algorithm is configured to satisfy the robust prior distribution (continuity constraint) chosen in step 2. The robust constraint is implemented using a highly efficient Kalman filter-Kalman smoother procedure that computes a quadratic approximation to the prior distribution defined by the continuity constraint. The high temporal resolution of the robust spectral analysis algorithm is achieved by using the Kalman filter algorithm to fuse information across adjacent time. That is, the spectral estimate at time t is used to compute the update at time t+1. Moreover, the contribution of the spectral estimate at time s<t+1 to the update at time t+1 decays exponentially as a function of t+1−s. In other words, spectral estimates that are closer in time to the spectral estimate at t+1 are given more weight than those that are further in time. This is a more principled approach to spectral estimation than windowing which does not impose a continuity constraint. Notice that our temporal continuity constraint is placed on the spectral components. This is, different from spectral estimation using finite-order autoregressive processes where the continuity constraint is placed on the coefficients of the autoregressive process.

Moreover, information obtained using the harmonic pursuit approach described could be used to obtain estimates of other parameters of interest, such as computation of higher-order spectra, phase amplitude modulation, or indices in relation to anesthetic effects. For instance, harmonic information generated could be combined to provide sparse estimates of higher order spectra, such as the bispectrum. The harmonic components might also be used as part of an algorithm to estimate phase-amplitude modulation, using the frequencies identified from the harmonic pursuit algorithm, as well as the harmonic amplitudes and phase. The harmonic components might also be used as an input for later processing for a characterization of overall level of anesthesia, summarized by a single scaled number (e.g., an index between 0 and 100). In this manner, information obtained or computed from high resolution spectral estimates, provided using methods accordance with the present disclosure, may be utilized in determining a current of future brain state of subject.

### Analysis of Spectral Resolution.

Determining the frequency resolution of a given estimator is a central question in spectral estimation. In order to characterize the resolution for non-parametric spectral estimators, the properties of the so-called taper that is applied to the data can be studied. For instance, the multitaper spectral estimator uses the discrete prolate spheroidal sequences as tapers, which are known to have very small side-lobes in the frequency domain. From the recursive form of the Fixed Interval Smoother, it is not evident how the robust estimator fits into the tapering framework of non-parametric spectral estimators. In what follows, we show how the spectral resolution of the robust spectral estimator can be characterized.

Before proceeding with the analysis, first consider the periodogram estimate. Recall the compact form of the forward model y=Fx+v, where F is given in equation (10). Assume for convenience that the window length W is an integer multiple of the number K of discrete frequencies, i.e. W=rK for some integer r, so that Fₙ=F₁, for all n. The periodogram maps the data y to the estimate:

$$\hat{x} := \frac{1}{rW} {F}^{H} y , \qquad ( 19 ) ;$$

from which the spectrum ∥\hat{x}∥₂ of the data can be computed. In other words, the rows of (1/rW)F^{H} form a bank of filters, which is formed of sliding windows of the Fourier basis on the interval [1,W]. The side-lobes of these filters determine the spectral resolution.

### Robust Spectral Estimation as a Filter Bank

The following proposition characterizes the equivalent filter banks corresponding to the robust spectral estimator:

Let W=rK for some integer r, so that Fₙ=F₁, for all n and let F be as defined in equation (10). Let Q^{(∞)} be the element-wise limit point of Q^{(l)}. Moreover, suppose that N is large enough so that L_{N|N} converges to its steady-state value denoted by Σ^{(∞)} and that each iteration of fixed interval smoother is initialized using the steady-state value of Σ_{N|N} in the previous iteration. Then, the estimate of the robust spectral estimator is given by:

$$\begin{matrix}{lim}_{𝓁 \to ∞} {\hat{x}}^{( 𝓁 )} = {GF}^{H} y ; \\ where & ( 20 ) \\ G := ( \begin{matrix}{Λ}^{0} Γ & {Λ}^{1} Γ & {Λ}^{2} Γ & … & {Λ}^{N - 1} Γ \\ {Λ}^{1} Γ & {Λ}^{0} Γ & {Λ}^{1} Γ & … & {Λ}^{N - 2} Γ \\ {Λ}^{2} Γ & {Λ}^{1} Γ & {Λ}^{0} Γ & … & {Λ}^{N - 3} Γ \\ ⋮ & ⋮ & ⋮ & ⋱ & ⋮ \\ {Λ}^{N - 1} Γ & {Λ}^{N - 2} Γ & {Λ}^{N - 3} Γ & … & {Λ}^{0} Γ\end{matrix} ) ; & ( 21 )\end{matrix}$$

with Λ:=τ^{(∞)}(Σ^{(∞)}+Q^{(∞)})⁻¹ and

γ:=(Σ^{(∞)}+Q^{(∞)})|I−rW((Σ^{(∞)}+Q^{(∞)})⁻¹+rWI)⁻¹|

Proof 2. Consider the Fixed Interval Smoother at the l^{th} iteration of the robust spectral estimator. By expanding the estimate \hat{x}_{n|N} in terms of (yₗ)ₗ₌₁^{N}, it is not hard to show that:

$${\hat{x}}_{n \vert N} = {\sum }_{n = 1}^{n - 1} [ {\prod }_{m = s}^{n - 1} ( I - {K}_{m} {F}_{m} ) ] {K}_{s} {y}_{s} + {K}_{n} {y}_{n} + {\sum }_{s = n + 1}^{N} [ {\prod }_{m = n}^{s} {B}_{m} ] {K}_{s} {y}_{s} ; \qquad ( 22 )$$

with Bₘ=Σ_{m|m}Σ_{m-1|m}⁻¹. Also, I−KₘFₘ=Σ_{m|m}Σ_{m+1|m}⁻¹. In the steady state, Σ_{m|m}=Σ^{(∞)} and Σ_{m|m-1}=Σ^{(∞)}+Q^{(∞)} for all m. Hence, the expression for \hat{x}_{t|N} simplifies to:

$${\hat{x}}_{n \vert N} = {\sum }_{s = 1}^{N} {Λ}^{[ s - n ]} Γ {F}_{s}^{H} {y}_{s} ; \qquad ( 23 )$$

with Λ and Γ as defined in the statement of the proposition. Expressing the above expression in compact form gives the statement of the proposition.

Proposition 2 states that the spectral estimate at window n is a tapered version of the Fourier transform of the data, where the taper at window s is given by Λ^{|s-n|}Γ. This can be viewed as an exponentially decaying taper in the matrix form, since the eigenvalues of Λ are bounded by 1. To illustrate this observation, assume that Q^{(∞)}=qI for some positive constant q. Then, it is not hard to verify that Γ=γI and Λ=αI, for some positive constants 0<γ≦1 and α>0. Then, the equivalent sliding taper applied to the data is given by the exponential taper αγ^{|s|}.

The rows of GF^{H} form a filter bank whose output is equivalent to that of the robust spectral estimator at windows n=1, 2, . . . , N. As mentioned earlier, the advantage of the weighting factor G is twofold. First of all, the weighting shapes the Fourier basis by an effectively exponential taper for higher side-lobe suppression. Secondly, the choice of Q^{(∞)} from the data, determines the gain of each filter. That is, the filters corresponding to the large (small) elements of Q^{(∞)} are likely to have a high (low) gain. Therefore, the shaping of the filters is determined by the data itself. Note that given Q^{(∞)}, Σ^{(∞)} can be computed by numerically solving a Riccati equation, and form the matrix GF^{H}, the rows of which are the equivalent bank of filters corresponding to the robust spectral estimator at windows n=1, 2, . . . , N.

This characterization of the spectral resolution of the robust spectral estimator, as well as its interpretation as a filter bank, applies to the case of Q^{(∞)} independent of n (time). This holds for log-prior f₁(•) and its associated Q^{(∞)}, which is the element-wise limit of equation (17). The element-wise limit of equation (18), which corresponds to log-prior f₂(•), is not independent of n. However, equation (22) is quite general and adopts a similar form once the appropriate substitutions are made. Thus, our attention can be restricted to Q^{(∞)} independent of n in order to convey the key ideas.

Thus, for f(•)=f₁(•), the estimate \hat{x} from the robust spectral estimator is given by:

$${lim}_{𝓁 \to ∞} {\hat{x}}^{( 𝓁 )} = {GF}^{H} y ; \qquad ( 24 )$$

where G is a weighting matrix, and is only a function of the window size W and Q^{(∞)}:=lim_{l→∞}Q^{(∞)}. The rows of GF^{H} form a filter bank whose output is equivalent to that of the robust spectral estimator at windows n=1, 2, . . . , N. As shown above, the advantages of the weighting matrix G are two-fold and detailed above and the shaping of the filters is determined by the data itself.

Equation (24) provides an ex post prescription to analyze the resolution and leakage of the robust spectral estimate. That is, given W and Q^{(∞)}, the matrix GF^{H} can be formed, the rows of which are the equivalent bank of filters corresponding to the robust spectral estimator in windows n=1, 2, . . . , N.

The data, for example, simulated in the toy example can be used to compare the spectral estimates computed using harmonic pursuit to the spectrogram computed using the multitaper method. Harmonic pursuit gives the sparse, more compact, representation that is desirable to recover given the simulated data of equation (7). Thus, in some embodiments, harmonic pursuit advantageously requires less memory and processing power which may be particularly beneficial for implementation in physiological monitors with limited resources. Indeed, faithfully recovering the two tones as well as temporal modulation is achievable. In addition, the spectral estimate is significantly denoised, such as illustrated in FIGS. 3A-3D. Harmonic Pursuit is able to overcome the fundamental limits imposed by the classical uncertainty principle, namely, the spectral estimate exhibits high resolution both in time and in frequency.

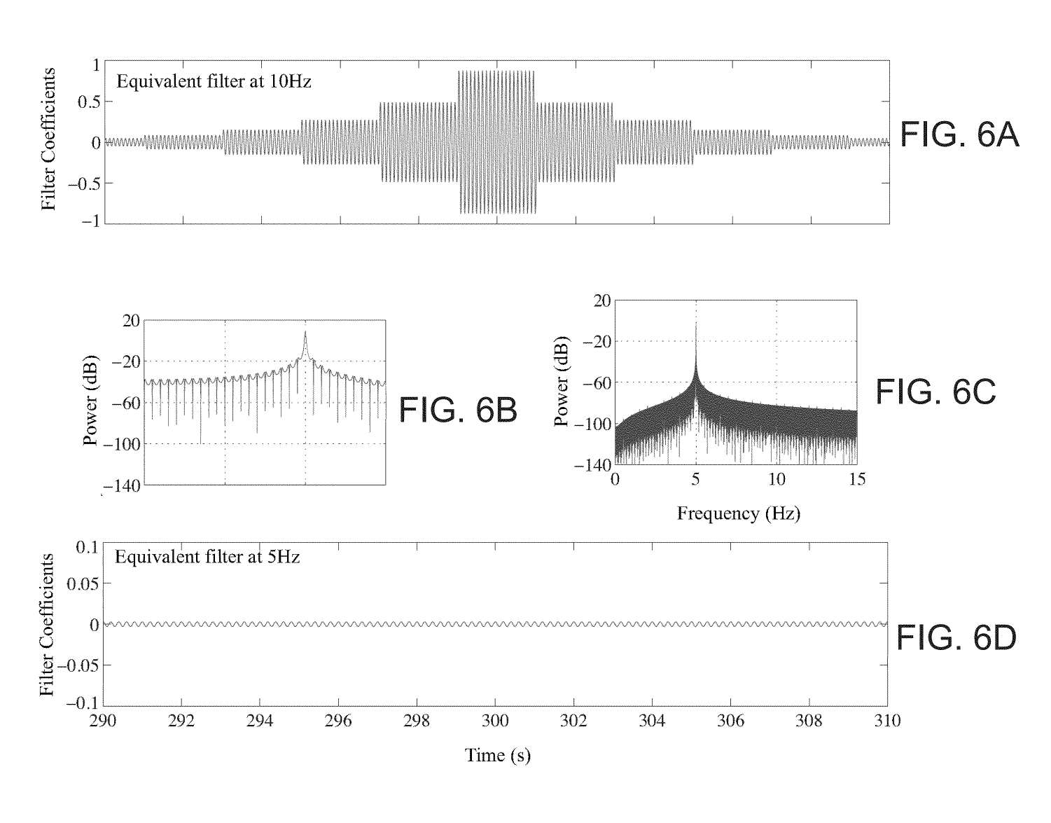

To illustrate this, the two filters that, when applied to the data from the toy example, reproduce the harmonic pursuit spectral estimates will be examined. Specifically, FIGS. 6A-6D show the equivalent filters corresponding to the harmonic pursuit estimator at frequencies 10 Hz and 5 Hz for t=300 s. The equivalent filter at 10 Hz corresponds to the component 10 cos⁸(2πf₁t) and, as explained relative to the toy example, resembles a 10 Hz oscillation which is exponentially decaying in a piece-wise constant fashion. The first side lobe is around 10.5 Hz, with a suppression of approximately 25 dB. The equivalent filter at 5 Hz, however, corresponds to a frequency that is not part of the signal. Hence, the peak gain is approximately 10 dB smaller than that of the 10 Hz filter. As a result, the 5 Hz component of the estimate is negligible.

This toy example demonstrates the potential of structured time-frequency representations used in the harmonic pursuit framework to go beyond the classical time-frequency resolution limits imposed by the uncertainty principle, and capture the dynamics of a non-stationary signal.

### Applications of Harmonic Pursuit to Neural Signal Processing

A harmonic pursuit analysis of EEG recordings provides various clinical benefits. First, the application of harmonic pursuit to this clinical setting can be illustrated by computing the spectrogram from frontal EEG data recorded from a patient during general anesthesia for a surgical procedure.

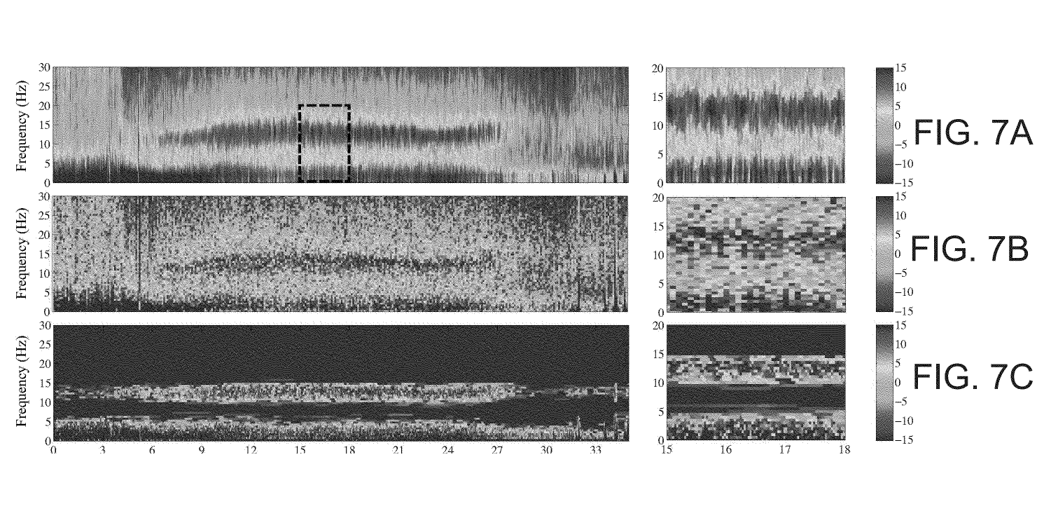

Consider FIGS. 7A through 7C. In this instance, the patient received a bolus intravenous injection of propofol at approximately 3.5 minutes, followed by a propofol infusion at 120 mcg/kg/min which was maintained until minute 27, when the case ended. When administered to initiate general anesthesia, propofol produces profound slow (<1 Hz) and delta (1 to 4 Hz) oscillations. With maintenance of general anesthesia, using propofol we observe an alpha oscillation (8 to 12 Hz) in addition to the slow and delta oscillations. The presence of the alpha oscillations along with the slow and delta oscillations is a marker of unconsciousness. The presence of these oscillations can be readily detected by analyzing the power in the relevant frequency bands using any systems and methods configured to do so. Developing a precise characterization of the dynamics of the dynamic properties of propofol is important for helping to define the neural circuit mechanisms of this anesthetic.

We computed the spectrogram for T=35 minutes of EEG data, sampled at a rate fs=250 Hz, using the multi-taper method with 1 s temporal resolution (as illustrated in FIG. 7A), a multi-taper method with 0.5 Hz frequency resolution (FIG. 7B), and the harmonic pursuit estimator with prior f₂(•) (FIG. 7C). The right panels of each figure show a zoomed-in view of the spectrogram from minute 15 to minute 18. For the harmonic pursuit analysis illustrated in FIG. 7C, W=500, N=1050 and α was selected by splitting the data into two sequences based on its even and odd times, respectively, and performing a form of two-fold cross validation. In other words, the harmonic pursuit estimate of the spectrum was assumed to be constant in windows of length 2 s. For each 2 s window of data, Fₙ is the 500×500 matrix, which is the Fourier basis for the discrete-time interval [(n−1)W+1,nW] for n=1, 2, . . . , N.

By setting the choice of tapers or the analysis window it is possible with the multitaper method to achieve either high frequency or high temporal resolution using the multitaper methods. In contrast, as in the toy example, harmonic pursuit achieves high temporal resolution, high spatial resolution, and performs significant denoising of the spectrogram. As a consequence of the simultaneous enhanced time-frequency resolution in the harmonic pursuit analysis, the slow and delta oscillations are clearly delineated during induction (minute 3.5), whereas during maintenance (minutes 5 to 27), the oscillations are strongly localized in the slow, delta and alpha bands. Furthermore, the denoising induced by harmonic pursuit creates a ≈30 dB difference between these spectral bands and the other frequencies in the spectrum. The Harmonic Pursuit analysis can achieve a more precise delineation of the time-frequency properties of the EEG under propofol general anesthesia, and anesthesia induced by other anesthetic compounds whose time-frequency properties have been characterized.

### A Harmonic Pursuit Analysis of Neural Spiking Activity.

Modulation of neuronal firing rates by brain rhythms has been well documented. During propofol-induced general anesthesia, the transition into and out of consciousness is characterized by abrupt changes both in average neuronal firing rates as well as their modulation by a low-frequency (≈0.5 Hz) oscillation. Local-field potentials (LFPs) are routinely recorded simultaneously with spike-train data. Typically, the spike-field coherence, a measure of phase synchronization between spike trains and LFPs, is used to quantify the modulation of firing rates by oscillations and its time course. Despite the prevalence of modulation of neuronal firing rates by brain rhythms, there are no principled approaches which use spike-train data alone, without recourse to LFPs, to extract oscillatory dynamics. The harmonic pursuit framework can be adapted to point-process data, and applied to neural spiking data acquired from a human subject during the induction of propofol general anesthesia.

Assume that the spike train from each of the neurons recorded is the realization of a point-process in the interval (0, T]. A point-process is an ordered sequence of discrete events that occur at random points in continuous time. For a neural spike train, the elements of the sequence give the times in (0, T] when the membrane potential of a neuron crosses a pre-determined threshold. That is to say the elements of the sequence give times when the neuron emits a spike. A point-process is fully characterized by its conditional intensity function (CIF). In our notation, the binary time-series yₜε{0,1}, t=1, . . . , T, represents the discrete-time point-process data. Denote by 0≦λₜ<∞, t=1, . . . , T, the sampled CIF of the point-process. Letting λₙ:=(λ_{(n-1)W+1)}, λ_{(n-1)W+2}, . . . , λ_{nW})′, as explained above in FIG. 5, the first step is the use of a harmonic forward model, in this case, a point-process harmonic forward model, which includes a harmonic parameterization of the CIF as follows:

log λₙ=Fₙxₙ, (25);

where log λₙ:=(log λ_{(n-1)W+1}, log λ_{(n-1)W+2}, . . . , log λ_{nW})′ for n=1, 2, . . . , N. Equation (25) is a time-frequency representation of the time-varying CIF of a point-process: xₙ, n=1, 2, . . . , N represent the signals modulating each of the oscillations (in Fₙ, n=1, 2, . . . , N) which form the CIF.

By solving the following MAP estimation problem:

$${max}_{{x}_{1} , … , {x}_{N}} {\sum }_{n = 1}^{N} {y}_{n}^{′} log {λ}_{n} - {1}_{w}^{1} {λ}_{n} = {f}_{2} ( {x}_{1} , {x}_{2} , … , {x}_{N} ) , \qquad ( 26 ) ;$$

(xₙ)ₙ₌₁^{N} can be estimated. In equation (26), 1_{W} is the vector of ones in [symbol] ^{W}, and under a generalized linear model (equation (25)). The objective function of equation (26) trades off the point-process log-likelihood with the log-prior in equation (13), which enforces sparsity in frequency and smoothness in time. Equation (26) is point-process version of harmonic pursuit (equation (15)). To this end, the solution \hat{x} for equation (26) can be computed as the limit of a sequence (\hat{x}^{(l)})ₗ₌₀^{∞} whose l^{th} element, l=1, . . . , ∞, is the solution to:

$${max}_{{x}_{1} , … , {x}_{N}} {\sum }_{n = 1}^{N} {y}_{n}^{′} {F}_{n} {x}_{n} - {1}_{w}^{′} {e}^{{F}_{n} {x}_{n}} - {\sum }_{k = 1}^{K} {\sum }_{n = 1}^{N} \frac{{( {x}_{n , k} - {x}_{n - 1 , k} )}^{2}}{2 ( {Q}_{n}^{( 𝓁 )} )} ; \qquad ( 27 )$$

where Qₙ^{(l)} is given in equation (18) and e^{F}^{ₙ}^{x}^{ₙ} is the [symbol] ^{W} vector with entries e^{(F}^{ₙ}^{x}^{ₙ}^{)w}, w=1, 2, . . . , W. Each iteration can be implemented efficiently using a point-process smoothing algorithm. It is not hard to prove that the conclusions of Theorem 1 regarding convergence also hold for the sequence generated using this algorithm. However, as stated above, convergence to a stationary point of the objective can be assured because f₂(•) is not convex.

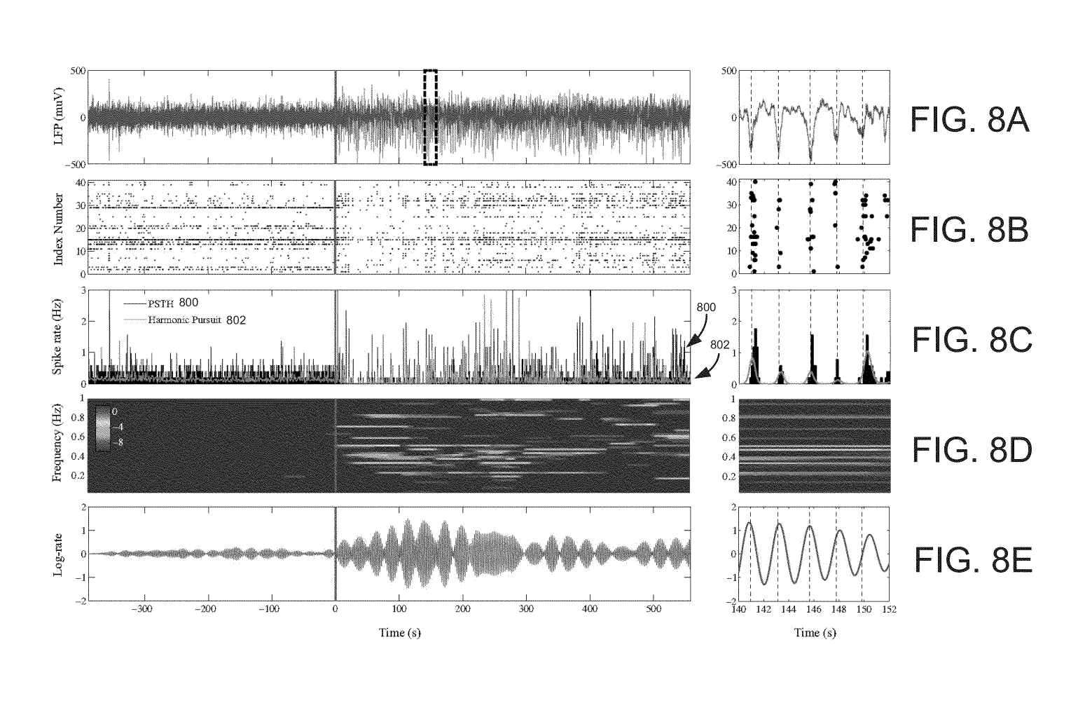

This algorithm was used to compute a spectral representation of the population rate function of 41 neurons recorded in a patient undergoing intracranial monitoring for surgical treatment of epilepsy using a multi-channel microelectrode array implanted in temporal cortex. Recordings were conducted during the administration of propofol for induction of anesthesia. FIGS. 8A through 8E are graphs that depicts the data collected during this experiment, as well as the results of analysis in accordance with the present disclosure. The right panels show the respective zoomed-in view of the left panels from t=140 s to t=152 s. FIGS. 8A and 8B show, respectively, the LFP activity and a raster plot of the neural spiking activity collected during the experiment. The bolus of propofol is administered at ≈0 s. Propofol-induced unconsciousness occurs within seconds of the abrupt onset of a slow (≈0.5 Hz) oscillation in the LFP. Moreover, neuronal spiking is strongly correlated to this slow oscillation, occurring only within a limited slow oscillation-phase window and being silent otherwise.

In Lewis L, et al. (“Rapid fragmentation of neuronal networks at the onset of propofol-induced unconsciousness.” Proceedings of the National Academy of Sciences 109:E3377-E3386, 2012) the authors extensively describe the experimental protocol under which these data were collected and further employ the data to elucidate the neurophysiological processes that characterize the transition into loss of consciousness (LOC) during propofol-induced general anesthesia. The findings of Lewis and colleagues rely primarily on the LFP activity recorded simultaneously with the neural spiking activity. In accordance with the present disclosure, using only the neural spiking activity, direct and precise evidence of the modulation of neural spiking activity by a slow oscillation during the transition into LOC under propofol-induced general anesthesia can be seen. FIG. 8C shows the firing rate estimates obtained using the standard peri-stimulus time histogram (PSTH) (black) with a bin size of 125 ms and the harmonic pursuit solution (red), respectively. In each 125 ms bin, the PSTH is the total number of spikes across all units divided by the product of the number of units and the bin size.

Consistent with the findings of Lewis and colleagues, the firing rate of the neurons reaches a maximum at the troughs of the slow ≈0.5 Hz oscillation in the LFP. The robust firing rate estimate from harmonic pursuit is much smoother. FIG. 8D shows the novel spectral decomposition of the firing rate of the cortical neurons in the range 0.05 Hz to 1 Hz during propofol-induced general anesthesia. For this analysis, fₛ=1 kHz, T=1000 s, W=125, and N=8000. In other words, the spectrum was assumed to be constant in windows of length 125 ms. For each 125 ms window of data, Fₙ is a 125×50 matrix obtained using 50 equally-spaced values in the range [0.05, 1] Hz. We constructed the observation vector yₙε [symbol] ¹²⁵ in each window by summing the spikes from all 41 units in each 1 ms bin. To select α, we split the 41 units randomly into two disjoint sets of 21 and 20 units, respectively, and performed two-fold cross-validation on this splitting.

This analysis reveals that the onset of LOC under propofol-induced general anesthesia in the patient is accompanied by the onset of a strong ≈0.45 Hz oscillation. Specifically, the onset of LOC can be readily detected by tracking the change in power in the spectrogram below 1 Hz. FIG. 8E shows the contribution of this ≈0.45 Hz oscillation to the log of the population firing rate (equation (25)). The extent to which this oscillation modulates the firing rate of cortical neurons at a resolution of 125 ms can be quantified. Prior to LOC (before ≈0 s), the analysis and FIG. 8E show that the slow oscillation does not contribute significantly to the firing rate of cortical neurons. However, during the recovery period, the slow oscillation increases the firing rate of cortical neurons by up to a factor of ≈3 above its local mean. Furthermore, FIG. 8E indicates that the ≈0.45 Hz component of the firing rate estimate from harmonic pursuit is 180 degrees out of phase with the LFP activity. In other words, following LOC, the troughs of the LFP activity coincide with the times at which the contribution of this oscillation to the firing rate of cortical neurons is maximum. In contrast to Lewis and colleagues, harmonic pursuit estimates the strong modulation of the firing rate of cortical neurons by a slow, ≈0.45 Hz, oscillation using only the neural spiking activity and without the LFP activity.

Robust spectral estimation for signals with structured time-frequency representations. As discussed, classical non-parametric spectral estimation methods use sliding windows with overlap to implicitly enforce temporal smoothness of the spectral estimate. This widely-adopted approach does not adequately describe processes with highly-structured time-frequency representations. As described above, a robust non-parametric spectral estimation paradigm is provided for batch time-series analyses, termed harmonic pursuit, that uses a Bayesian formulation to explicitly enforce smoothness in time and sparsity in frequency of the spectral estimate. Harmonic pursuit yields spectral estimates that are significantly denoised and have significantly higher time and frequency resolution than those obtained using the multitaper method. Computation of harmonic pursuit spectrogram estimates includes solving a high-dimensional optimization problem (equation (15) with substitutions from equations (14), (13), and (12)).

By using a relation between l₁ minimization, Gaussian scale mixture models, and the EM algorithm, computationally efficient IRLS algorithms are provided to carry out the spectral estimation. In the clinical applications discussed, these algorithms can be implemented with Kalman and point-process smoothing algorithms. The achievable spectral resolution can be characterized because the harmonic pursuit solution applies a time-varying, data-dependent filter bank to the observations (FIGS. 6A-6D).

Harmonic pursuit offers a principled alternative to existing methods, such as EMD, for decomposing a noisy time-series into a small number of harmonic components. In harmonic pursuit, this is handled using the regularization parameter a, which can be estimated from the time-series using cross-validation.

### Link of Harmonic Pursuit to Basis Pursuit Denoising, IRLS Algorithms, and Sparse Recovery.

This work builds on results in basis pursuit denoising (BPDN) and IRLS algorithms developed to compute sparse decompositions in noise-free systems. The BPDN algorithm has been used to demonstrate that static signals can be decomposed into sparse representations using finite dictionaries obtained by discretizing in the frequency domain, as described in Chen S S, Donoho D L, Saunders M A (1996) Atomic decomposition by basis pursuit, which is incorporated herein by reference in its entirety. Daubechies and colleagues showed under mild conditions that in the absence of noise, IRLS algorithms can recover sparse signals, as described in Daubechies I, DeVore R, Fornasier M, Gunturk C (2009) Iteratively reweighted least squares minimization for sparse recovery. Communications on Pure and Applied Mathematics 63:1-38., which is incorporated herein by reference in its entirety. The IRLS algorithm of Daubechies and colleagues can solve a BPDN-type optimization problem. We recently extended the work of Daubechies and colleagues to solve the problem of sparse recovery in the presence of noise, as described in Ba D, Babadi B, Purdon P, Brown E (2013) Convergence and stability of iteratively reweighted least squares algorithms, to appear in IEEE Transactions on Signal Processing., which is incorporated herein by reference in its entirety. Along with doing so, we broadened the family of IRLS algorithms. The IRLS algorithms derived from the penalty functions in equations (12) and (13) belong to the broader class of IRLS algorithms. A key insight applied here is that this broad class of IRLS algorithms can be used in the context of Bayesian estimation of state-space models to compute highly-structured spatio-temporal decompositions. Harmonic pursuit offers a new approach to robust spectral analysis of batch time series that is applicable to a broad range of problems.

The various configurations presented above are merely examples and are in no way meant to limit the scope of this disclosure. Variations of the configurations described herein will be apparent to persons of ordinary skill in the art, such variations being within the intended scope of the present application. For example, the above-described systems and methods may be utilized with other system, for example, as described in co-pending International Application No. PCT/US13/64852 and U.S. application Ser. No. 14/151,412, which are both incorporated herein by reference in their entirety. To this end, the above-described systems and methods may be used to determine a current state or predict a future state of a patient receiving a compound with anaesthetic or sedative properties.

Features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A system for monitoring a patient experiencing an administration of at least one drug having anesthetic properties, the system comprising:
at least one sensor configured to acquire physiological data from a patient;
a processor configured to:
(i) receive the physiological data from the at least one sensor;
(ii) assemble a time-frequency representation of signals from the physiological data;
(iii) apply a state-space model for the time-frequency representation of the signals to enforce spectral estimates that are smooth in time and sparse in a frequency domain;
(iv) iteratively adjust weightings associated with the spectral estimates to converge the data toward a desired outcome; and
(v) generate a report indicating a physiological state of the patient.

2. The system of claim 1 wherein the processor is further configured to use a Bayesian formulation to perform (iii).

3. The system of claim 1 wherein the processor is further configured to perform iteratively re-weighted least squares (IRLS) algorithm to perform (iv).

4. The system of claim 1 wherein the desired outcome includes determining a global maximum to a maximum a-posterior (MAP) estimation problem.

5. The system of claim 1 wherein the processor, to perform (iii), is further configured to impose a prior distribution on a time-frequency plane, which enforces spectral estimates that are smooth in time, and sparse in the frequency domain

6. The system of claim 1 wherein the report includes a spectrogram.

7. The system of claim 1 wherein the processor is further configured to perform a Bayesian estimation the state-space model to compute a highly-structured spatio-temporal decomposition with respect to the spectral estimates.

8. A system for monitoring a patient, the system comprising:
at least one sensor configured to acquire physiological data from a patient;
a processor configured to:
(i) receive the physiological data from the at least one sensor;
(ii) apply a spectral estimation framework that utilizes structured time-frequency representations defined by imposing, to the physiological data, a prior distributions on a time-frequency plane that enforces spectral estimates that are smooth in time and sparse in a frequency domain;
(iii) perform an iteratively re-weighted least squares algorithm to perform yield a denoised time-varying spectral decomposition of the physiological data; and
(iv) generate a report indicating a physiological state of the patient.

9. The system of claim 8 wherein the processor is further configured to assemble the physiological data into a time-series of signals and to decompose the time-series of signals into a small number of harmonic components to perform (iii).

10. The system of claim 9 wherein the processor is further configured use a regularization parameter estimated from the time-series of signals using cross-validation to decompose the time-series of signals into a small number of harmonic components

11. The system of claim 8 wherein the processor is further configured to perform iteratively re-weighted least squares (IRLS) algorithm to perform (iii).

12. The system of claim 8 wherein the processor is further configured to perform a Bayesian estimation a state-space model to compute the decomposition of the physiological data.

13. The system of claim 8 wherein the report includes a spectrogram.

14. The system of claim 8 further comprising an input configured to receive input about an administration of at least one drug having anesthetic properties to the patient.

15. A method of processing a time-series of data comprising:
applying a spectral estimation framework that utilizes structured time-frequency representations (x) of the time-series of data (y) defined by:
imposing, on the time-series of data, a prior distribution in a time-frequency plane that enforces spectral estimates that are smooth in time and sparse in the frequency domain;
determining, using an iteratively re-weighted least squares (IRLS) algorithm, spectral estimates that are maximum a posteriori (MAP) spectral estimates; and
generating a report indicating using the spectral estimates that are maximum a posteriori (MAP) spectral estimates.

16. The method of claim 15 wherein the report includes a spectrogram.

17. The method of claim 15 wherein the time-series of data includes electroencephalogram (EEG) data.

18. The method of claim 15 wherein the MAP spectral estimates are calculated using the following:
$${max}_{{x}_{1} , … , {x}_{N}} - {\sum }_{n = 1}^{N} \frac{1}{2 {σ}^{2}} {‖ {y}_{n} - {F}_{n} {x}_{n} ‖}_{2}^{2} - f ( {x}_{1} , {x}_{2} , … , {x}_{N} ) ;$$
$$where {( {F}_{n} )}_{l , k} := cos ( j 2 π ( ( n - 1 ) W + l ) \frac{k}{K} ) and$$
$${y}_{n} := {( {y}_{( n - 1 ) W + 1} , {y}_{( n - 1 ) W + 2} , … {y}_{nW} )}^{′}$$
for an interval of length W, n=1, 2, . . . N with
$$N := \frac{T}{W} ,$$
T as an integer multiple of W, σ as a constant, and l=1, . . . , ∞.

19. The method of claim 15 further comprising acquiring the time-series of data from at least one sensor coupled to a patient experiencing an administration of at least one drug having anesthetic properties.

20. The method of claim 19 wherein the at least one drug having anesthetic properties includes at least one of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil.
